(19) United States
(12) Patent Application Publication
(10) Pub. No.: US 2018/0161276 A1
(43) Pub. Date: Jun. 14, 2018

(54) TEMPERATURE-RESPONSIVE COMPOSITION, METHOD FOR FABRICATING, AND USE THEREOF

(71) Applicant: NATIONAL TSING HUA UNIVERSITY, HSINCHU (TW)

(72) Inventors: Hsing-Wen SUNG, HSINCHU (TW); Yu-Jung LIN, New Taipei City (TW); Nai-Wen CHI, New Taipei City (TW); Wei-Lin WAN, Hsinchu County (TW)

(21) Appl. No.: 15/603,419

(22) Filed: May 23, 2017

(30) Foreign Application Priority Data
Dec. 14, 2016 (TW) 105141456

(51) Int. Cl. A61K 9/16; A61K 33/00

(52) U.S. Cl. CPC A61K 9/1635; A61K 9/1617; A61K 33/00

(57) ABSTRACT

The present disclosure provides a temperature-responsive composition including a nitric oxide donor and a carrier. The nitric oxide donor is a pH-dependent material, and the carrier is provided for carrying the nitric oxide donor with an effective amount. The carrier includes a protective agent and an organic protic acid. A transition temperature of the temperature-responsive composition is larger than or equal to 28° C. and less than or equal to 37° C.

### RELATED APPLICATIONS

This application claims priority to Taiwan Application Serial Number 105141456, filed Dec. 14, 2016, which is herein incorporated by reference.

### BACKGROUND

### Technical Field

The present disclosure relates to a temperature-responsive composition. More particularly, the present disclosure relates to a temperature-responsive composition, method for fabricating and use the same capable of prolonging the half-life of the nitric oxide.

### Description of Related Art

Nitric oxide (NO) has a higher diffusion rate, penetrates through a cell membrane easily and is unstable so as to be an intercellular or intracellular signal molecule. That is, nitric oxide can be an effective signal for adjusting respiratory system, digestive system and endocrine and metabolic system of an advanced organism. Recently, nitric oxide is further provided for treating cardiovascular system, immune system, cancer or skeletal system and even can accelerate a healing of a wound so as to be attended extensively.

Functions of nitric oxide for bone metabolism have been studied recently when nitric oxide is utilized as a therapeutic gas. With the extension of human life, osteoporosis becomes second most-common disease according to the analysis of the world health organization, and a serious bone fracture even results in death. It is known that cytokines, such as interleukin-1 (IL-1), tumor necrosis factor (TNF) and interferon-γ (IFN-γ), will affect the activity of nitric oxide synthase (NOS) of a bone tissue. When the concentration of the accumulated nitric oxide achieves a proper level, the formation of osteoclasts and the function of mature osteoclasts will be inhibited. Otherwise, the function of osteoblasts will be affected and even the bone resorption of the osteoclasts will be prompted when the concentration of the accumulated nitric oxide is insufficient.

In addition, a half-life of nitric oxide is quite short due to a self-oxidation of nitric oxide or a reaction between nitric oxide and erythrocyte in plasma or ceruloplasmin. Thus, nitric oxide is limited for medical applications.

### SUMMARY

The present disclosure provides a temperature-responsive composition. The temperature-responsive composition includes a nitric oxide donor and a carrier. The nitric oxide donor is a pH-dependent material, and the carrier includes a protective agent and an organic proton acid for carrying the nitric oxide donor with an effective amount. Furthermore, a transition temperature of the temperature-responsive composition is larger than or equal to 28° C. and less than or equal to 37° C.

The present disclosure further provides a method for fabricating the temperature-responsive composition as mentioned above. The method includes providing an oil phase solution, providing a water phase solution, performing an emulsification and collecting the temperature-responsive composition. In the step of providing the oil phase solution, the oil phase solution includes the nitric oxide donor, the protective agent and the proton acid. Then, the step of performing the emulsification is achieved by mixing the oil phase solution and the water phase solution for forming the temperature-responsive composition. Finally, the temperature-responsive composition will be collected.

The present disclosure further provides a method for treating indications for nitric oxide therapy, and the method includes administering an effective amount of the above-mentioned temperature-responsive composition to a subject suffered from the indications for nitric oxide therapy.

### BRIEF DESCRIPTION OF THE DRAWINGS

The present disclosure can be more fully understood by reading the following detailed description of the embodiment, with reference made to the accompanying drawings as follows:

FIG. 1 is a structural schematic view showing a temperature-responsive composition according to the present disclosure;

FIG. 2 is a flow chart showing a method for fabricating a temperature-responsive composition according to the present disclosure;

FIG. 3 is an operational schematic view showing a fabrication of a temperature-responsive composition according to a first embodiment of the present disclosure;

FIG. 4A are images of the temperature-responsive composition according to the first embodiment of the present disclosure;

FIG. 4B is a relation between days of storage and nitric oxide (NO) level of the temperature-responsive composition according to the first embodiment of the present disclosure;

FIG. 5A are ultrasonic images obtained under different conditions, wherein the image (a) and the image (b) show a process of producing nitric oxide by using diethylenetriaine(DETA)-NONOate, and the image (c) and the image (d) show a process of producing nitric oxide by using the temperature-responsive composition according to the first embodiment of the present disclosure;

FIG. 5B is a structural schematic view showing a bubble produced in FIG. 5A;

FIG. 5C are images showing a simulation process by using diethylenetriaine-NONOate and capric acid to simulate a nitric oxide production through the temperature-responsive composition according to the first embodiment of the present disclosure;

FIG. 6A shows an anti-degradation ability of nitric oxide produced by using diethylenetriaine-NONOate; and

FIG. 6B shows an anti-degradation ability of nitric oxide produced by using the temperature-responsive composition according to the first embodiment of the present disclosure.

### DETAILED DESCRIPTION

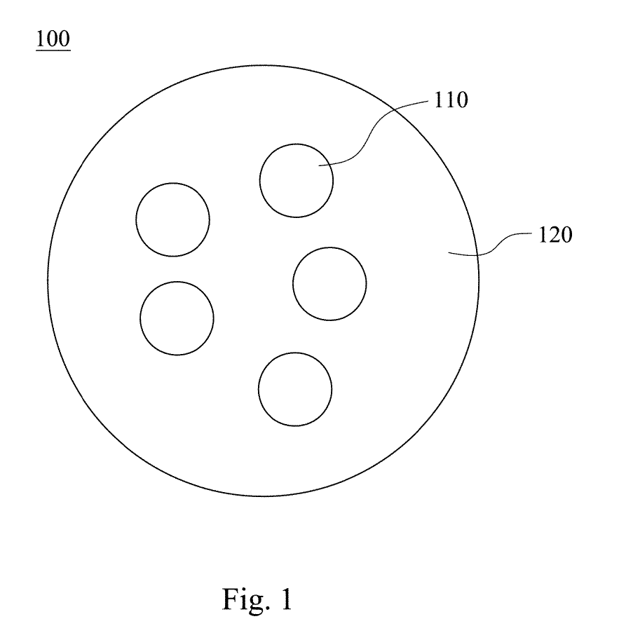

Please refer to FIG. 1, which is a structural schematic view showing a temperature-responsive composition 100 according to the present disclosure. As shown in FIG. 1, the temperature-responsive composition 100 can be a sphere microparticle and include a nitric oxide donor 110 and a carrier 120. In particular, the nitric oxide donor 110 can be a pH dependent material thus to be decomposed quickly for producing nitric oxide by changing the acidity of the environment. Moreover, the carrier 120 is provided for carrying the nitric oxide donor 110, and the nitric donor 110 is preferably evenly dispersed in the carrier 120.

The carrier 120 includes a protective agent and an organic proton acid. In particular, the organic proton acid can be provided for adjusting the acidity of the environment of the nitric oxide donor 110 and has functions, such as amphiphilic and phase-changeable, so as to stabilize nitric oxide-containing bubbles. More particularly, the organic proton acid can be capric acid or lauric acid.

The protective agent can prevent the nitric oxide donor 110 from decomposing quickly, which is caused by the organic proton acid. The selection and the content of the protective agent of the carrier 120 can be further adjusted corresponding to the organic proton acid. In particular, a transition temperature of the temperature-responsive composition 100 can be larger than or equal to 28° C. and less than or equal to 37° C. Thus, the temperature-responsive composition 100 is a solid at room temperature, such as 25° C. Then, the temperature-responsive composition 100 will change its phase due to a body temperature of the human body in the following application. More particularly, the transition temperature of the temperature-responsive composition 100 is between a first melting point of the protective agent and a second melting point of the organic proton acid by adjusting a mixing ratio of the protective agent and the organic proton acid of the carrier 120. When the organic proton acid of the carrier 120 is capric acid with a melting point of 31.6 OC and the protective agent of the carrier 120 is octadecane with a melting point between 28° C. and 30° C., for example, the protective agent and the organic proton acid can be mixed at a weight ratio of the protective agent to the organic proton acid of 1:0.25 to 1:4. Accordingly, the transition temperature of the temperature-responsive composition 100 can be set up to be larger than or equal to 28° C. and less than or equal to 37° C. It is noted that the protective agent can be but not limited to nonadecane, icosane, 1-Tetradecanol or hexadecan-1-ol when the organic proton acid is capric acid. Moreover, the organic proton acid can be lauric acid with a melting point between 44° C. and 46° C., and the protective agent can be selected from an alkane or alkanol with a lower melting point, such as C11-C16 alkane or C11-C12 alkanol, to allow the transition temperature of the temperature-responsive composition 100 to be less than or equal to 37° C.

Accordingly, except the temperature-responsive composition 100 of the present disclosure can be stored easily at a low temperature, such as 4° C., it also can be changed from a solid state to a melted state due to the body temperature of the human body in the following medical application. Thus, the nitric oxide donor 110 will further react with the organic proton acid of the melted carrier 120 to produce nitric oxide-containing bubbles in plasma.

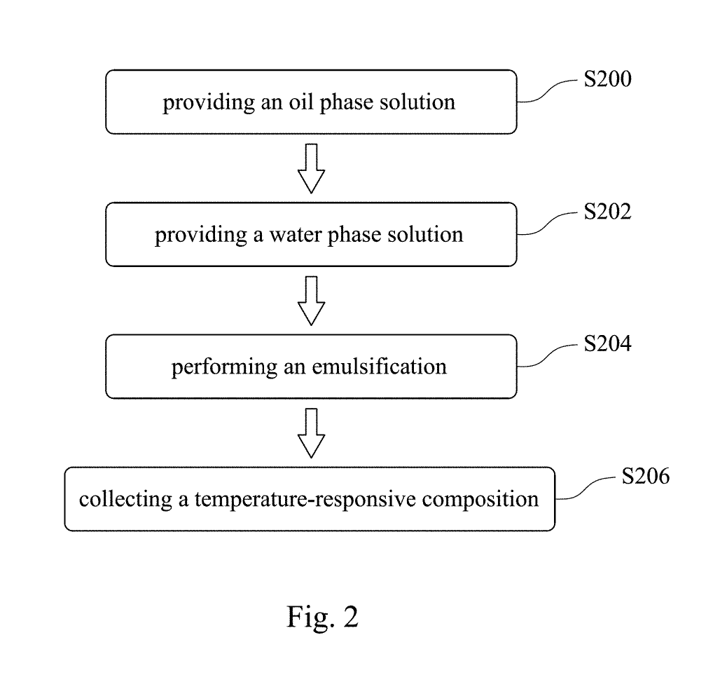

Please refer to FIG. 2, which is a flow chart showing a method for fabricating a temperature-responsive composition according to the present disclosure. The method includes Step S200, Step S202, Step S204 and Step S206.

In details, Step S200 and Step S202 provide an oil phase solution and a water phase solution, respectively. In order to avoid the nitric oxide donor from reacting with the organic proton acid quickly, the nitric oxide donor is mixed with the protective agent at first for obtaining a first solution. The first solution is then mixed with a second solution containing the organic proton acid for obtaining the oil phase solution.

In Step S204, the oil phase solution obtained from Step S200 is mixed with the water phase solution obtained from Step S202 to perform an emulsification for forming the temperature-responsive composition.

In Step S206, the temperature-responsive composition can be collected by steps of filtering, washing and drying.

Accordingly, the temperature-responsive composition, which is fabricated from the above-mentioned steps, can be utilized as one of drug ingredients for treating indications for nitric oxide therapy, for example, osteoporosis. Furthermore, the drug can include a pharmaceutically acceptable salt.

The temperature-responsive composition has been described as mentioned above. In the following, a first embodiments and a second embodiment will be further provided to illustrate the above-mentioned temperature-responsive composition, the method for fabricating thereof and the accompanied effect in details. However, the present disclosure is not limited thereto.

### Temperature-Responsive Composition and Method for Fabricating Thereof

### 1st Embodiment

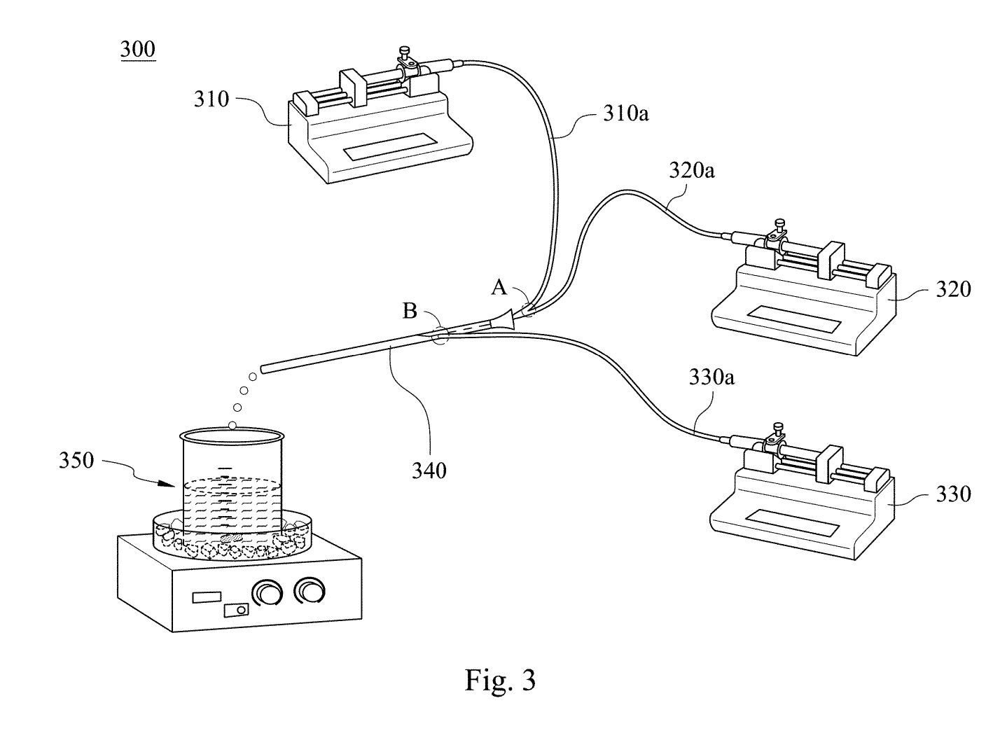

Please refer to FIG. 3 in conjunction with FIG. 2, in which FIG. 3 is an operational schematic view showing a fabrication of a temperature-responsive composition according to a first embodiment of the present disclosure. As shown in FIG. 3, the method for fabricating the temperature-responsive composition in the present disclosure is performed in a microfluidic system 300.

The microfluidic system 300 includes a first infusion pump 310, a second infusion pump 320, a third infusion pump 330, a transmission unit 340, a collecting unit 350 and three tubes 310a, 320a, 330a for connecting the first infusion pump 310, the second infusion pump 320 and the third infusion pump 330, respectively, to the transmission unit 340.

In the 1st embodiment, the transmission unit 340 is set up by polishing a 26G needle with a gauge diameter of 0.45 mm and then embedding the 26G needle into an 18G stainless steel needle (1.2 mm). Then, the first solution and the second solution are prepared separately. In particular, 5 mg of a nitric oxide donor is evenly dispersed in a protective agent to form a first solution. More particularly, the protective agent of the 1st embodiment is 99% octadecane, the nitric oxide donor is diethylenetriaine-NONOate, and the second solution contains 98% capric acid.

The first solution and the second solution are loaded into the tube 310a and the tube 320a, respectively, by the first infusion pump 310 and the second infusion pump 320. Then, the first solution and the second solution are mixed uniformly at an intersection A of the tube 310a and the tube 320a for forming an oil phase solution. Preferably, a weight ratio of the protective agent (that is, octadecane) to the organic proton acid (that is, capric acid) in the oil phase solution is 1:3 so that the transition temperature of the temperature-responsive composition of the 1st embodiment is 29.5±0.7° C.

In the 1st embodiment, a polyvinyl alcohol (PVA) solution is provided as a water phase solution. The polyvinyl alcohol solution is loaded into the tube 330a by the third infusion pump 330 and mixed with the oil phase solution at an intersection B of the tube 330a and the transmission unit 340 to perform an emulsification for forming the temperature-responsive composition, and the temperature-responsive composition will flow into the collecting unit 350 along with the polyvinyl alcohol solution. In addition, the tube 310a, the tube 320a, the tube 330a and the transmission unit 340 can be placed in a warm bath to allow the above-mentioned steps to be performed at a predetermined temperature. Preferably, the predetermined temperature ranges from 55° C. to 60° C.

A filter paper is finally provided to remove the polyvinyl alcohol solution and filter out the temperature-responsive composition. The temperature-responsive composition is then washed by a phosphate buffered saline (PBS) solution and air-dried at 4° C. for the following analysis and application.

It is noted that diethylenetriaine-NONOate is water-soluable and decomposed easily by capric acid. Thus, diethylenetriaine-NONOate is dispersed in octadecane at first in the 1st embodiment and loaded into an individual channel for reducing the probability of the above-mentioned decomposition. Moreover, it is favorable to perform the emulsification of Step S204 in the microfluidic system 300 for forming the temperature-responsive composition with a uniform particle size. However, the emulsification of Step S204 also can be performed by stirring or homogenizing.

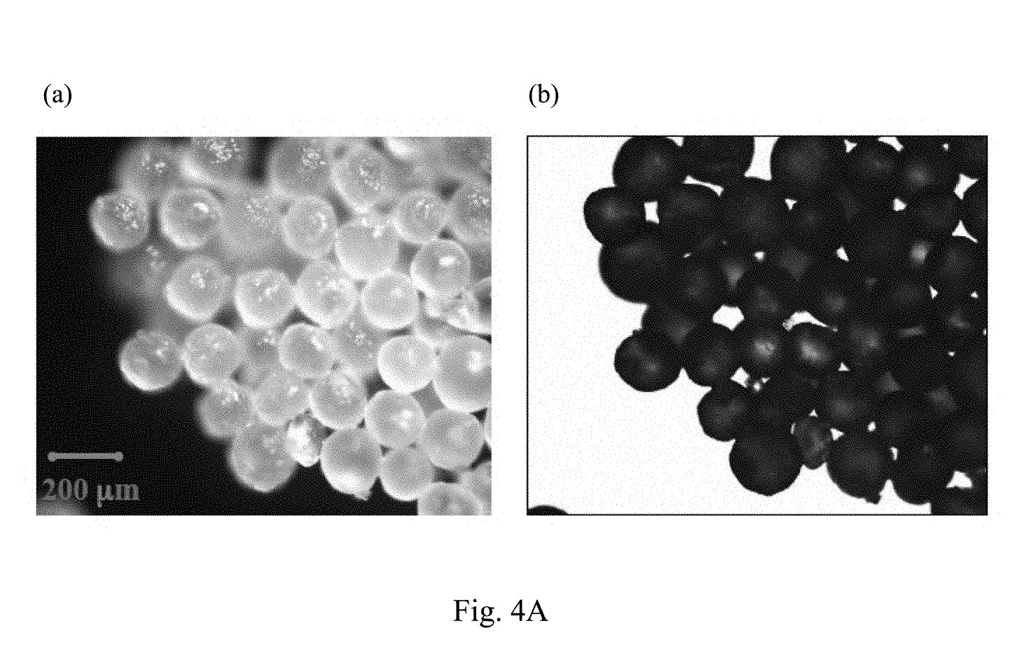

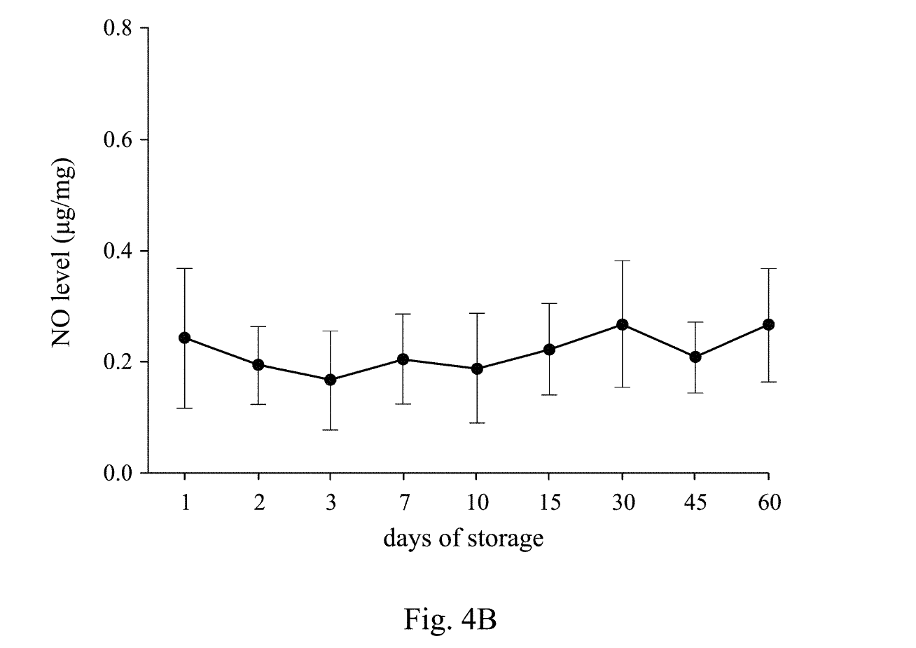

Please refer to FIG. 4A and FIG. 4B. FIG. 4A are images of the temperature-responsive composition according to the 1st embodiment of the present disclosure, and FIG. 4B is a relation between days of storage and NO level of the temperature-responsive composition according to the first embodiment of the present disclosure. In details, FIG. 4A (a) and FIG. 4A (b) are images of the above-mentioned temperature-responsive composition obtained by an optical microscopy from a top light source and a bottom light source, respectively. FIG. 4B shows the stability of the temperature-responsive composition at a low temperature, such as 4° C.

In FIG. 4B, nitric oxide is easily degraded to a stable metabolite (that is, nitrite) so that NO level can be detected by reacting a Griess reagent with nitrite. In details, different concentrations of diethylenetriaine-NONOate are added into a hydrochloric acid solution, separately, for producing nitric oxide. After reacting with the Griess reagent, an absorption intensity of 540 nm can be detected by a multi-mode microplate reader so as to obtain a standard curve of a relation between the absorption intensity and NO level. Then, 10 mg of the temperature-responsive composition to be tested is placed in a phosphate buffered saline solution and then moved to an oven with a setting temperature of 37° C. Accordingly, the temperature-responsive composition changes from a solid state to a melted state so as to release diethylenetriaine-NONOate. After reacting with the Griess reagent, NO level can be obtained through the above-mentioned standard curve.

In particular, the temperature-responsive composition is a sphere microparticle as shown in FIG. 4A (a) and FIG. 4A (b), and diethylenetriaine-NONOate, octadecane and capric acid are mixed uniformly. The particles of FIG. 4A have an average size of 228 μm, and the nitric oxide level, which is the amount of nitric oxide can be released, per milligram of the temperature-responsive composition is 0.38 μg. In addition, the temperature-responsive composition of the present disclosure can be stored for over 60 days under the above-mentioned condition.

### 2nd Embodiment

A temperature-responsive composition of the 2nd embodiment and a method for fabricating thereof are similar to the 1st embodiment. However, a weight ratio of octadecane to capric acid in a carrier is different from the 1st embodiment so that a transition temperature of the temperature-responsive composition herein is different from that of the 1st embodiment. The weight ratio of octadecane to capric acid in the carrier, the corresponding transition temperature of the temperature-responsive composition and NO level per milligram of the temperature-responsive composition are listed in Table 1.

TABLE 1

| | Weight ratio of | Transition | |
|---|---|---|---|
| | octadecane to capric | temperature | NO level |
| | acid in carrier | (° C.) | (μg/mg) |
| 2nd | 35:1 | 30.8 ± 0.3 | 0.38 ± 0.15 |
| embodiment | | | |

As shown in Table 1, the amount of nitric oxide released by the temperature-responsive composition is higher than that of the 1st embodiment when the weight ratio of octadecane to capric acid in the carrier is 3.5:1. That is, the temperature-responsive composition of the 2nd embodiment also has a good coverage for diethylenetriaine-NONOate. Moreover, the promotion of the transition temperature of the temperature-responsive composition in the 2nd embodiment is favorable for further application.

### Use of Temperature-Responsive Composition

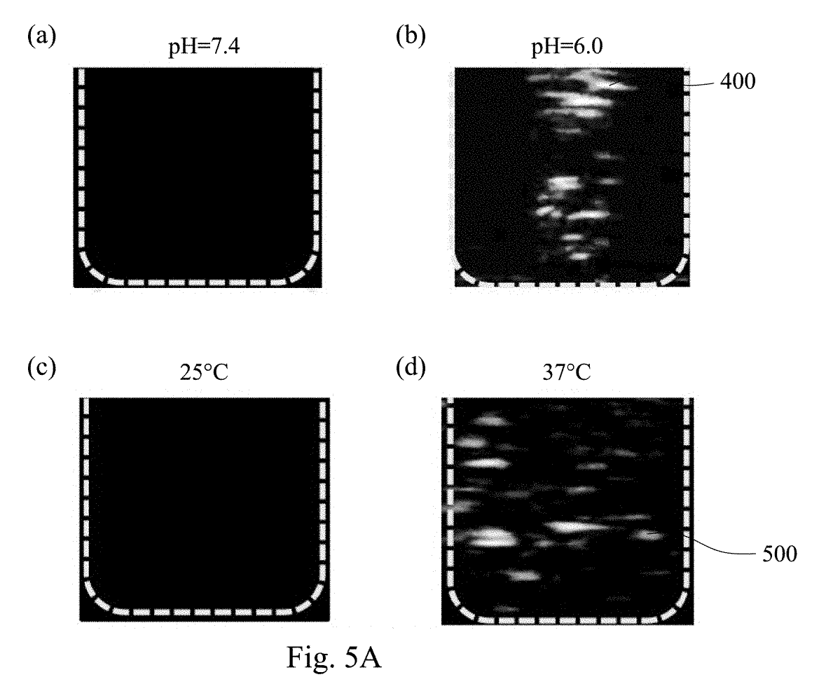

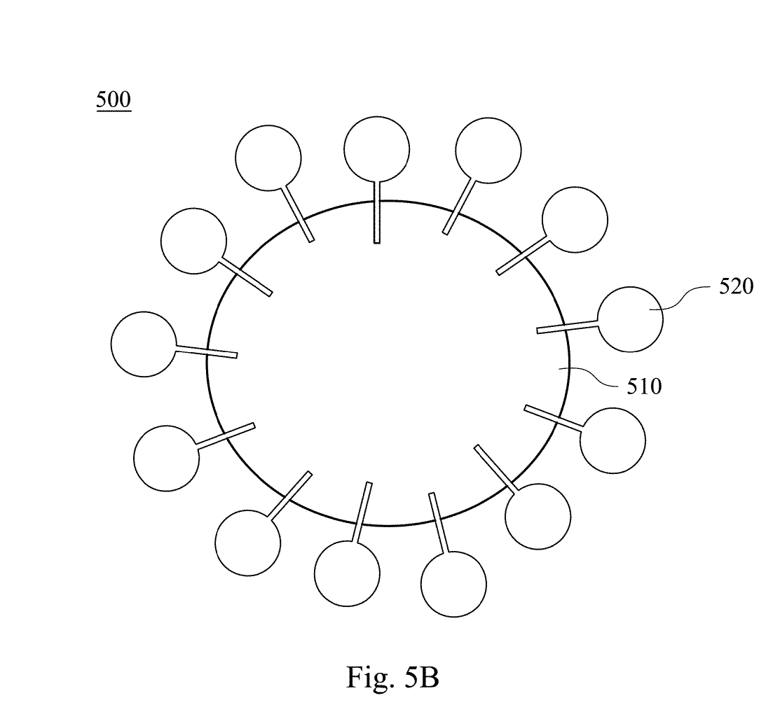

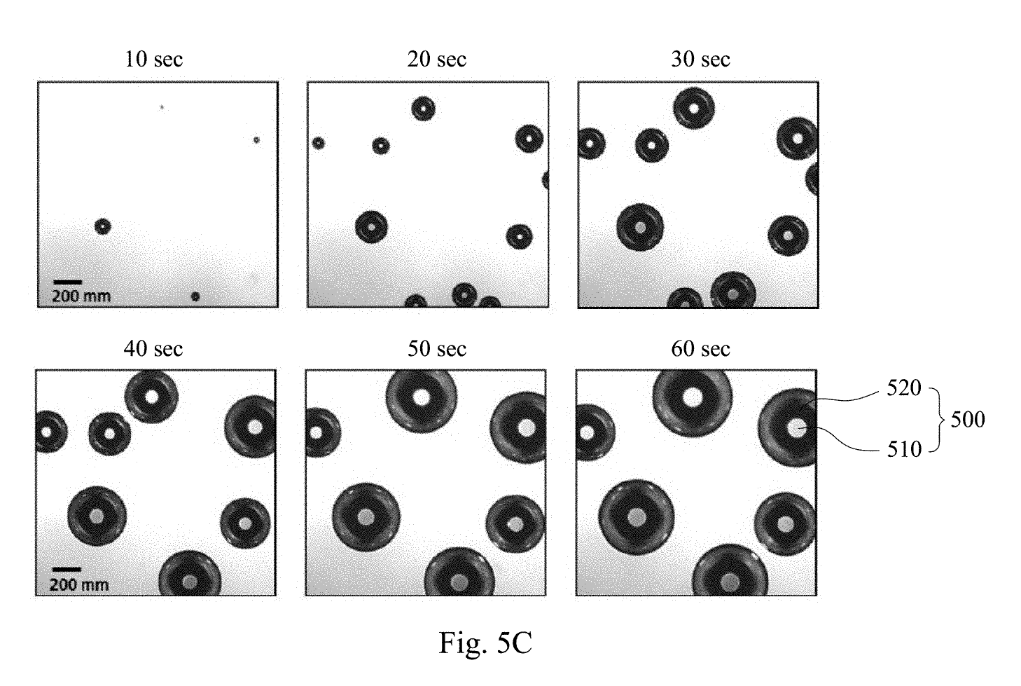

Please refer to FIG. 5A, which are ultrasonic images obtained under different conditions. In details, the image (a) and the image (b) of FIG. 5A show a process of producing nitric oxide by using diethylenetriaine-NONOate, and the image (c) and the image (d) show a process of producing nitric oxide by using the temperature-responsive composition according to the first embodiment of the present disclosure. In addition, FIG. 5A (a) and FIG. 5A (b) are obtained in an operating environment of a phosphate buffered saline solution. However, the temperature-responsive composition of the present disclosure is existed in the oil phase so that FIG. 5A (c) and FIG. 5A (d) is obtained in an operating environment of silicone oil for preventing the occurrence of reflection interferences resulted from the changes of the intermedium.

As mentioned above, diethylenetriaine-NONOate is a pH-dependent material. Accordingly, diethylenetriaine-NONOate is decomposed quickly for producing nitric oxide when the acidity of the operating environment changes from 7.4 to 6.0. Thus, a plurality of nitric oxide-containing bubbles 400 will be produced in the phosphate buffered saline solution as shown in FIG. 5A (a) and FIG. 5A (b). On the other hand, diethylenetriaine-NONOate is carried by the carrier in the 1st embodiment, and the transition temperature of the temperature-responsive composition is less than 37° C., that is, 29.5±0.7° C. Thus, the temperature-responsive composition of the 1st embodiment is existed in a solid state under the operating environment with a temperature of 25° C. so that there is no bubble produced as shown in FIG. 5A (c). When the temperature of the operating environment is increased to 37° C. for simulating the body temperature of the human body, the temperature-responsive composition changes from the solid state to a melted state so as to allow diethylenetriaine-NONOate to contact with capric acid for producing nitric oxide as shown in a formula (1):

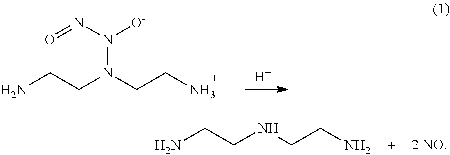

Accordingly, there are nitric oxide-containing bubbles 500 produced in the silicone oil as shown in FIG. 5A (d).

Please refer to FIG. 5B, which is a structural schematic view showing a bubble 500 produced in FIG. 5A. In details, nitric oxide 510 produced by the temperature-responsive composition of the 1st embodiment is covered and stabilized by capric acid 520 of the carrier at the same time. Accordingly, it prevents the produced nitric oxide from contacting plasma directly for extending the half-life of the produced nitric oxide when the temperature-responsive composition is applied for treating osteoporosis. Moreover, capric acid is favorable for inhibiting bone cell differentiation.

Please refer to FIG. 5C, which are images showing a simulation process by using diethylenetriaine-NONOate and capric acid to simulate a nitric oxide production through the temperature-responsive composition according to the first embodiment of the present disclosure. In details, free diethylenetriaine-NONOate, hydrochloric acid and capric acid labelled by a cyanine dye Cy5 are added into a phosphate buffered saline solution to simulate a nitric oxide production through the temperature-responsive composition according to the 1st embodiment of the present disclosure, in which hydrochloric acid is provided as a proton source, the cyanine dye is provided for observing if capric acid covers the produced nitric oxide. As shown in FIG. 5C, there are nitric oxide-containing bubbles produced in the phosphate buffered saline solution gradually with the increase of reaction time. The above-mentioned bubbles are all covered by capric acid labelled with the cyanine dye.

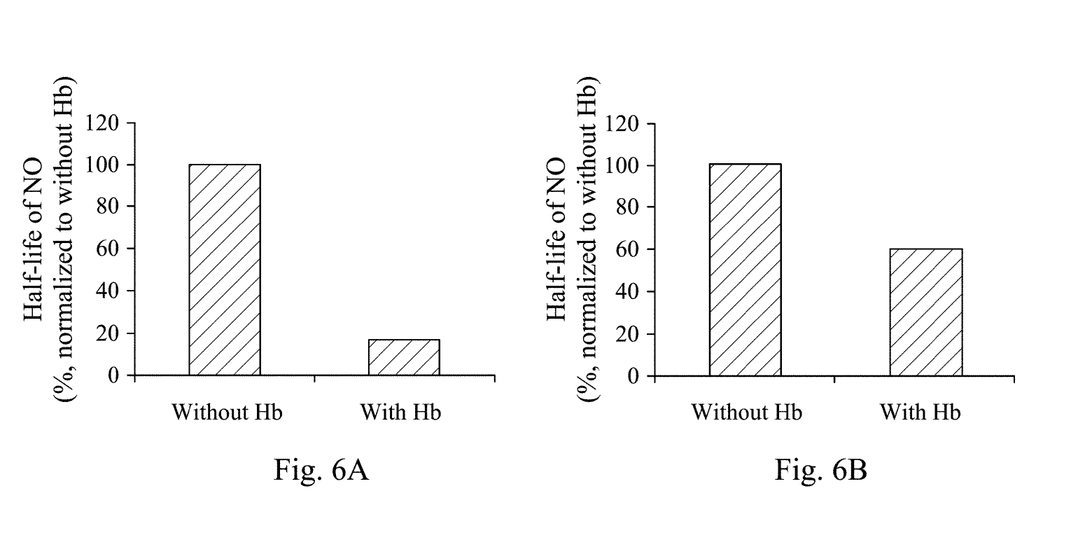

As mentioned above, the half-life of nitric oxide is quite short due to the reaction between nitric oxide and erythrocyte in plasma or ceruloplasmin. Please refer to FIG. 6A and FIG. 6B. FIG. 6A shows an anti-degradation ability of nitric oxide produced by using diethylenetriaine-NONOate, and FIG. 6B shows an anti-degradation ability of nitric oxide produced by using the temperature-responsive composition 510 according to the first embodiment of the present disclosure.

Compared to the half-life of nitric oxide, which is produced by using diethylenetriaine-NONOate in the absence of hemoglobin (Hb), the half-life of nitric oxide, which is produced by using diethylenetriaine-NONOate in the presence of hemoglobin, has a degradation of 85% as shown in FIG. 6A. However, the half-life of nitric oxide, which is produced by using the temperature-responsive composition of the 1st embodiment in the present disclosure in the presence of hemoglobin, only has a degradation of 40% as shown in FIG. 6B. That is, it proves that applying the temperature-responsive composition of the present disclosure can extend the half-life of the produced nitric oxide.

To sum up, osteoporosis can be prevented from several aspects by using the temperature-responsive composition of the present disclosure. First, nitric oxide produced by reacting the nitric oxide donor with the organic proton acid after phase changing is capable of adjusting the bone cell growth and differentiation and inhibiting the function of the osteoclasts. Second, the organic proton acid can provide protons for stabilizing the produced nitric oxide and inhibiting the differentiation of the osteoclasts. Finally, a loading frequency of nitric oxide can be reduced substantially by extending the half-life of the produced nitric oxide so as to increase the convenience for preventing and treating osteoporosis.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A temperature-responsive composition, comprising:
a nitric oxide donor being a pH-dependent material; and
a carrier comprising a protective agent and an organic proton acid for carrying the nitric oxide donor with an effective amount;
wherein a transition temperature of the temperature-responsive composition is larger than or equal to 28° C. and less than or equal to 37° C.

2. The temperature-responsive composition of claim 1, wherein the protective agent is C14-C20 alkane or C11-C16 alkanol.

3. The temperature-responsive composition of claim 1, wherein the nitric oxide donor is diethylenetriaine-NONOate.

4. The temperature-responsive composition of claim 1, wherein the proton acid is capric acid or lauric acid.

5. The temperature-responsive composition of claim 1, wherein the protective agent and the proton acid of the carrier are contained in a weight ratio of 1:0.25 to 1:4.

6. The temperature-responsive composition of claim 1, wherein the temperature-responsive composition is a sphere microparticle with a particle size ranged from 5 μm to 300 μm.

7. The temperature-responsive composition of claim 1, wherein nitric oxide level per milligram of the temperature-responsive composition ranges from 0.23 μg to 0.53 μg.

8. A method for fabricating the temperature-responsive composition of claim 1, comprising the following steps:
providing an oil phase solution, wherein the oil phase solution comprises the nitric oxide donor, the protective agent and the proton acid;
providing a water phase solution;
performing an emulsification by mixing the oil phase solution and the water phase solution for forming the temperature-responsive composition; and
collecting the temperature-responsive composition.

9. The method of claim 8, wherein the step of providing the oil phase solution comprises the following steps:
providing a first solution by mixing the nitric oxide donor and the protective agent;
providing a second solution, wherein the second solution comprises the proton acid; and
mixing the first solution and the second solution.

10. The method of claim 8, wherein the protective agent is C14-C20 alkane or C11-C16 alkanol.

11. The method of claim 8, wherein the nitric oxide donor is diethylenetriaine-NONOate.

12. The method of claim 8, wherein the proton acid is capric acid or lauric acid.

13. The method of claim 8, wherein the protective agent and the proton acid in the step of providing the oil phase solution are contained in a weight ratio of 1:0.25 to 1:4.

14. The method of claim 8, wherein the water phase solution comprises a polyvinyl alcohol solution.

15. The method of claim 8, wherein the method is performed in a microfluidic system.

16. A method for treating indications for nitric oxide therapy, comprising:
administering an effective amount of the temperature-responsive composition of claim 1 to a subject suffered from the indications for nitric oxide therapy.

17. The method of claim 16, wherein the indications for nitric oxide therapy comprise osteoporosis, wound healing, vasodilatation or partial tissue inflammation.
